(19) United States
(12) Patent Application Publication
(10) Pub. No.: US 2026/0161271 A1
(43) Pub. Date: Jun. 11, 2026

(54) MEDICAL SYSTEMS, DEVICES, AND METHODS FOR PROVIDING USER-ADJUSTABLE OUTPUT DATA LAYOUTS

(71) Applicant: Boston Scientific Scimed, Inc., Maple Grove, MN (US)

(72) Inventors: Mildred ANTWI-NSIAH, Brighton, MA (US); Geoffrey Douglas ASHTON, Hudson, MA (US)

(73) Assignee: Boston Scientific Scimed, Inc., Maple Grove, MN (US)

(21) Appl. No.: 19/184,096

(22) Filed: Apr. 21, 2025

Related U.S. Application Data
(60) Provisional application No. 63/637,508, filed on Apr. 23, 2024.

(51) Int. Cl. G06F 3/0484; A61B 1/00; G06F 3/04817

(52) U.S. Cl. CPC G06F 3/0484; A61B 1/0004; A61B 1/0005; G06F 3/04817

(57) ABSTRACT

A computing system includes a memory and a processor to detect a connection of one or more medical devices to the computing system, each of the one or more medical devices configured to provide image or video data to the computing system, cause display of a user interface associated with generation of a custom layout for presenting the image or video data, receive one or more inputs via the user interface that define the custom layout, including one or more of: a number of the one or more image or video tiles, a size of the one or more image or video tiles, or an association of the one or more medical devices with the one or more image or video tiles, and store the custom layout such that the image or video data is provided for display in accordance with the custom layout to one or more display devices.

### CROSS-REFERENCE TO RELATED APPLICATIONS

This application claims the benefit of priority to U.S. Provisional Application No. 63/637,508, filed on Apr. 23, 2024, which is incorporated by reference herein in its entirety.

### TECHNICAL FIELD

The disclosure relates generally to medical systems, devices, and methods. More specifically, aspects of the disclosure pertain to medical systems, devices, and methods for providing user-adjustable output data layouts.

### BACKGROUND

Endoscopic or other internal medical imaging systems capture images and/or video(s) of a target site during a medical procedure. In some aspects, the captured images and/or video(s) are displayed (e.g., contemporaneously or at a later time) on one or more displays or monitors. For example, the medical imaging systems may be connected to a computing system that is configured to receive and process the images and/or video(s) for output to the displays or monitors. The displays may be in the operating or treatment room, or may be external or remote from the operating treatment room. The displays may present images and/or videos from more than one source (e.g., multiple cameras or other imaging devices (e.g., X-rays, MRIs, CT, fluoroscopy, etc.)), patient information, operator information, device or system information, and/or other information for the procedure. However, the selection and/or presentation of the information may not be easily and/or quickly customizable for each user or operator's preference, the type of procedure, etc.

The systems, devices, and methods of this disclosure may rectify some of the deficiencies described above or address other aspects of the art.

### SUMMARY

In some examples, a computing system may be configured to be removably coupled to one or more medical devices. The computing system may include at least one memory and at least one processor. The at least one memory may be configured to store instructions. The at least one processor may configured to execute the instructions to: detect the one or more medical devices that are removably coupled to the computing system, each of the one or more medical devices may be configured to provide image or video data to the computing system; cause display of a user interface associated with generation of a custom layout for presenting the image or video data provided by the one or more medical devices, the user interface may include one or more image or video tiles configurable for inclusion in the custom layout; receive one or more inputs via the user interface that define the custom layout, where the one or more inputs may include one or more of: a number of the one or more image or video tiles, a size of the one or more image or video tiles, or an association of the one or more medical devices with the one or more image or video tiles; and store the custom layout such that, when the image or video data is received from the one or more medical devices, the image or video data may be provided for display in accordance with the custom layout to one or more display devices associated with the computing system.

The computing system may include one or more of the following features. The computing system may include a touch screen configured to display the user interface and receive the one or more inputs. Each of the one or more image or video tiles may include a plurality of frame edges, and each of the plurality of frame edges may be movable to adjust a size and/or shape of the image or video tile. The user interface may include a number of screens control portion that is manipulatable to adjust a number of the one or more image or video tiles included in the custom layout. The number of screens control portion may include an add screen icon and a reduce screen icon that are each selectable to adjust the number of the one or more image or video tiles included in the custom layout. Each of the one or more image or video tiles may include an input selection icon that may include an indication of the association of a medical device, from the one or more medical devices removably coupled to the computing system, with the respective image or video tile. The association may cause image or video data received from the medical device to be displayed via the respective image or video tile in the custom layout. Each input selection icon further may include a selection change icon that is selectable to adjust which medical device, from the one or more medical devices removably coupled to the computing system, is associated with the respective image or video tile.

Each of the one or more image or video tiles may be movable, via the user interface, to adjust a position of the respective image or video tile included in the custom layout. The user interface may further be configured to display a plurality of saved layouts of the one or more image or video tiles. The user interface may further include patient and/or user information. The user interface may include an active procedure screen that may include the custom layout selected for a procedure to be performed using the one or more medical devices, and one or more of: an image capture icon that may be selectable to capture one or more still images received from the one or more medical devices, a record icon that may be selectable to record one or more videos received from the one or more medical devices, and a stream icon that may be selectable to stream one or more aspects of the procedure.

The one or more medical devices may include one or more imaging or video devices, and one or more illumination devices controllable by the computing system. The user interface may be configured to display an illumination control section that may receive user inputs to adjust a brightness of the one or more illumination devices. The illumination control section may include a slider that is movable within a track to adjust the brightness of the one or more illumination devices. The computing system may be coupled to each of the one or more medical devices via a wired connection through an umbilicus. The computing system may be coupled to the one or more display devices via a wired connection or via a wireless connection.

In another aspect, a medical computing system may include a display, at least one memory, and at least one processor. The display may be for displaying a plurality of user interfaces, and may be configured to receive one or more inputs via the plurality of user interfaces. The at least one memory may be configured to store instructions. The at least one processor may be configured to execute the instructions to: detect one or more input sources that are coupled to the medical computing system, each of the one or more input sources may be configured to provide image or video data to the medical computing system; cause display of a user interface, from the plurality of user interfaces, associated with generation of a custom layout for presenting image or video data provided by the one or more input sources, the user interface may include one or more image or video tiles configurable for inclusion in the custom layout; receive one or more inputs via the user interface that define the custom layout, wherein the one or more inputs may include one or more of: a number of one or more image or video tiles, a size of the one or more image or video tiles, or an association of the one or more input sources with the one or more image or video tiles; and store the custom layout such that, when the image or video data may be received from the one or more input sources, the image or video data may be provided for display in accordance with the custom layout to one or more display devices associated with the computing system.

The medical computing system may include one or more of the following features. Each of the one or more image or video tiles may include a plurality of L-shaped corners. Each of the plurality of L-shaped corners may be movable to adjust a size and/or shape of the corresponding image or video tile. Each of the one or more custom layouts may include a number of screens control portion that may be adjustable to adjust a number of image or video tiles on the custom layout. The number of screens control portion may include an add screen icon and a reduce screen icon that may be each selectable to adjust the number of image or video tiles on the custom layout.

In yet another aspect, a method performed by a computing system that is removably connectable to a medical device for user during a medical procedure may include: receiving an input, via a user interface displayed on a display of the computing system, to enter a custom layout mode, where, in the custom layout mode, the user interface may display one or more image or video tiles that each may include a plurality of frame edges that may be manipulatable to adjust a size of each of the image or video tiles; receiving, via the user interface, one or more inputs of a number of screens corresponding to the number of image or video tiles; receiving, via the user interface, one or more inputs to adjust the size of one or more of the image or video tiles; and receiving, via the user interface one or more inputs to save the size and/or shape of the one or more image or video tiles as a customized layout

The method may further include receiving one or more inputs to adjust an arrangement of the one or more image or video tiles on the user interface.

It may be understood that both the foregoing general description and the following detailed description are exemplary and explanatory only and are not restrictive of the invention, as claimed. As used herein, the terms “comprises,” “comprising,” “includes,” “including,” “has,” “having,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate examples of this disclosure and, together with the description, serve to explain the principles of the disclosure.

FIG. 1 depicts an example medical system, including at least one medical device, for which layouts of image, video, and/or data displays may be customized or otherwise adjusted via one or more user interfaces.

FIGS. 2A-2E depict various exemplary screens of a user interface.

FIG. 3 depicts another exemplary screen of the user interface.

FIG. 4A-4C depict various customizable layout screens of the user interface.

FIG. 5 depicts yet another exemplary screen of the user interface.

FIG. 6 depicts an example process for selection and/or customization of one or more image, video, and/or data layouts.

FIG. 7 depicts an example computing device.

### DETAILED DESCRIPTION

As briefly mentioned above, one or more medical devices can be connected to a computing system to perform one or more different medical procedures. The one or more medical devices may capture and/or provide various images, videos, and/or data to be displayed on one or more displays. The one or more medical procedures may also require various information (e.g., user/operator name, patient information, date, time, duration, etc.), which may also be displayed on the one or more displays. Additionally, different users may have different display preferences, for example, for different types of procedures and/or for different numbers of image, video, and/or data displays. As such, various aspects of this disclosure are directed to medical systems, devices, and methods for customizing and/or adjusting one or more displays of various image(s), video(s), data, and/or other information for one or more procedures.

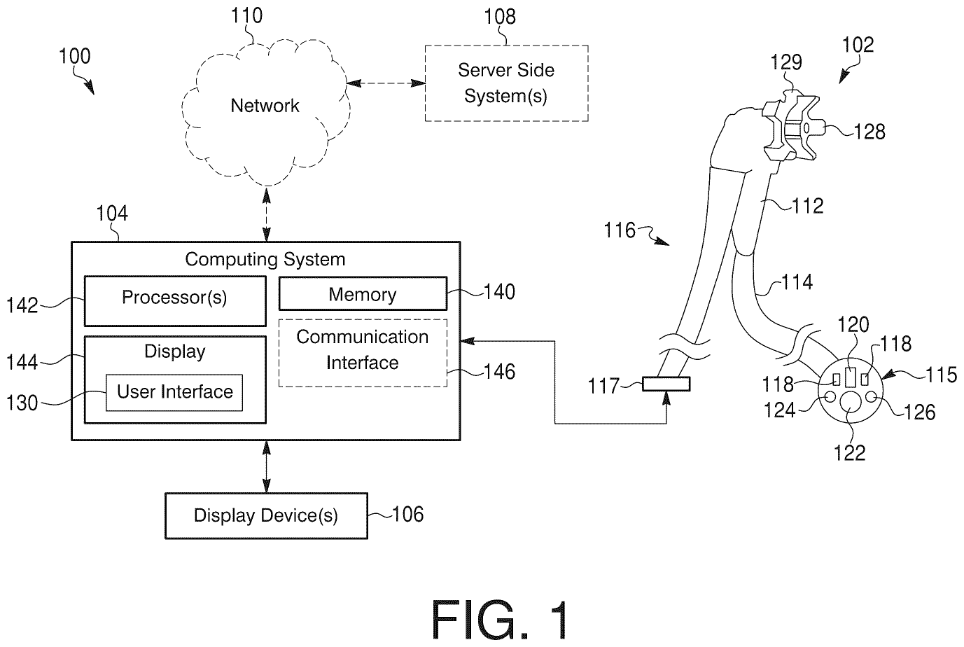

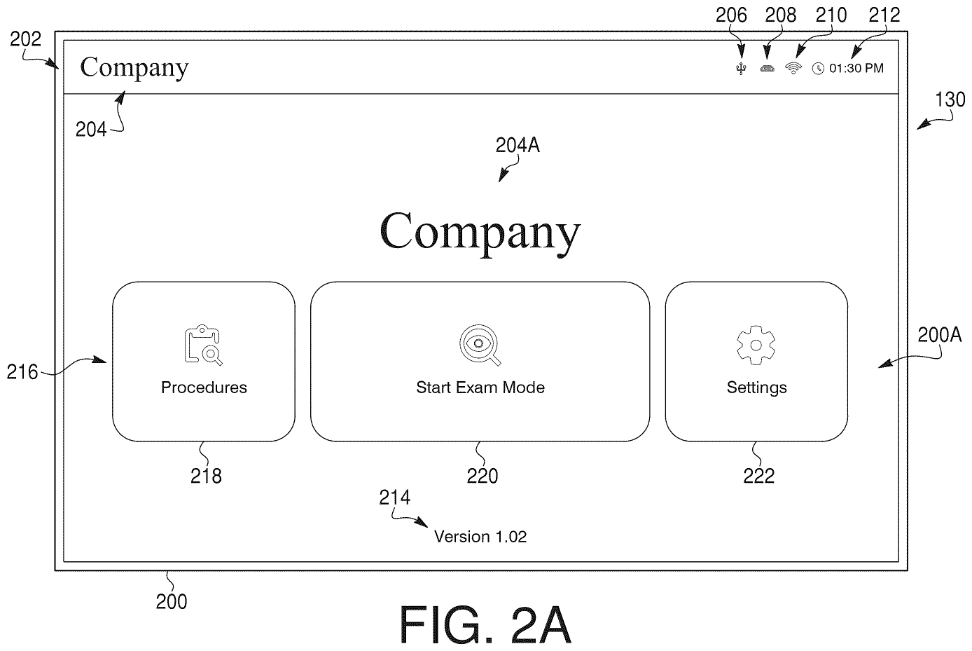

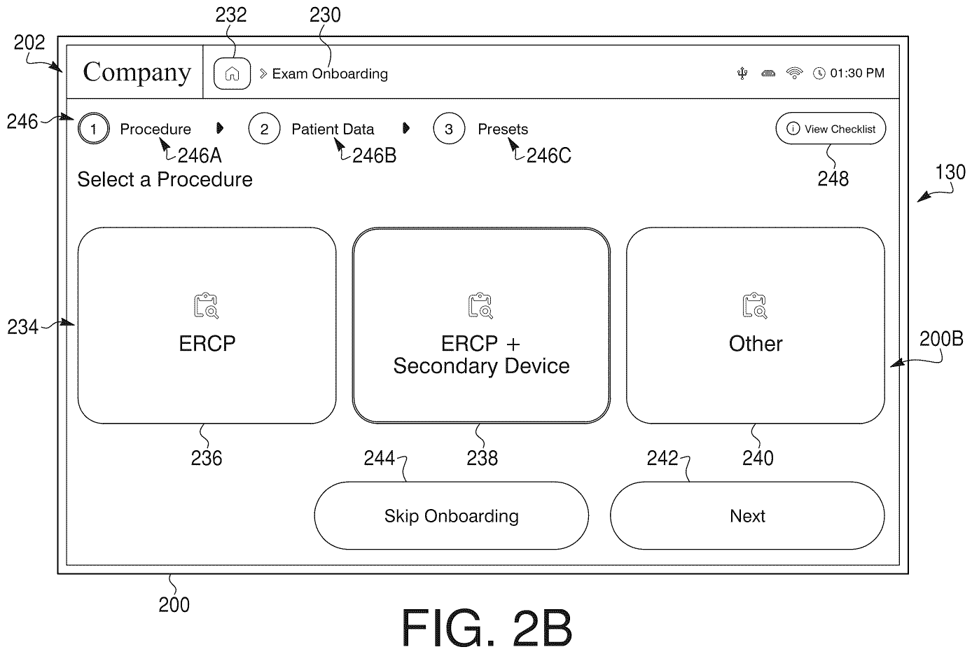

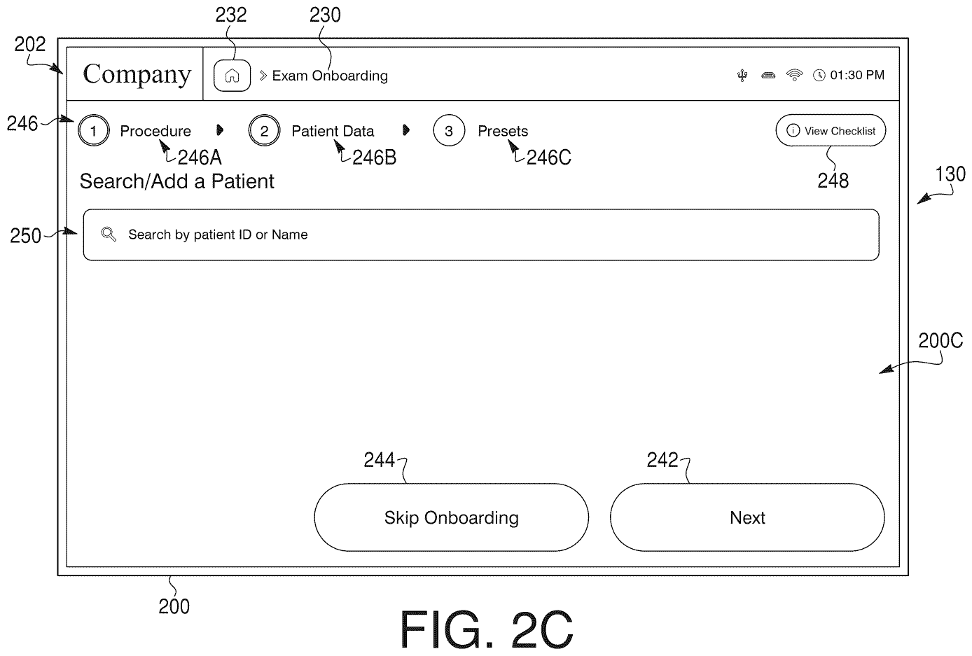

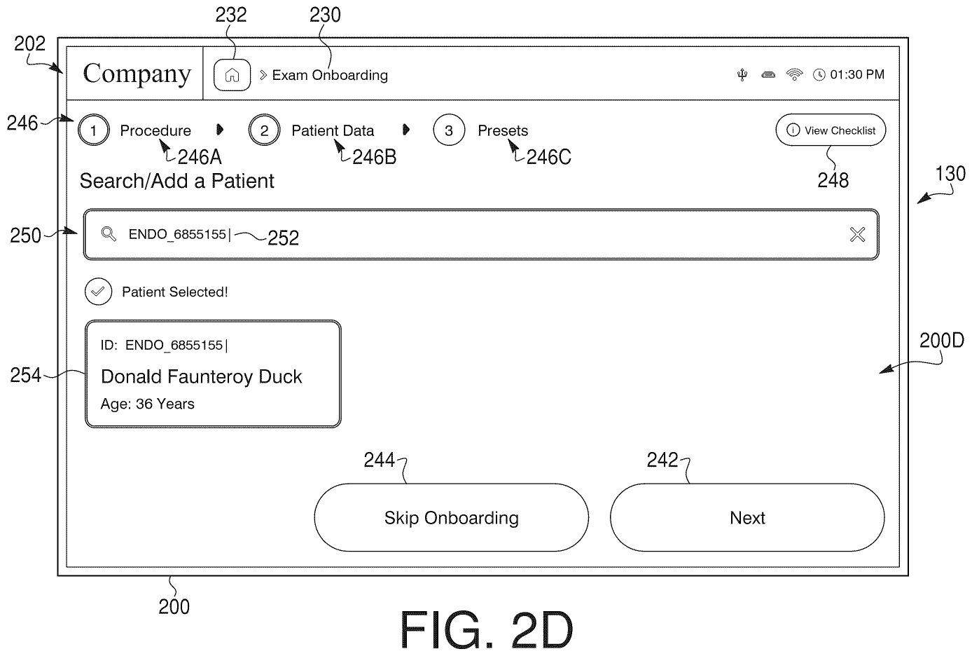

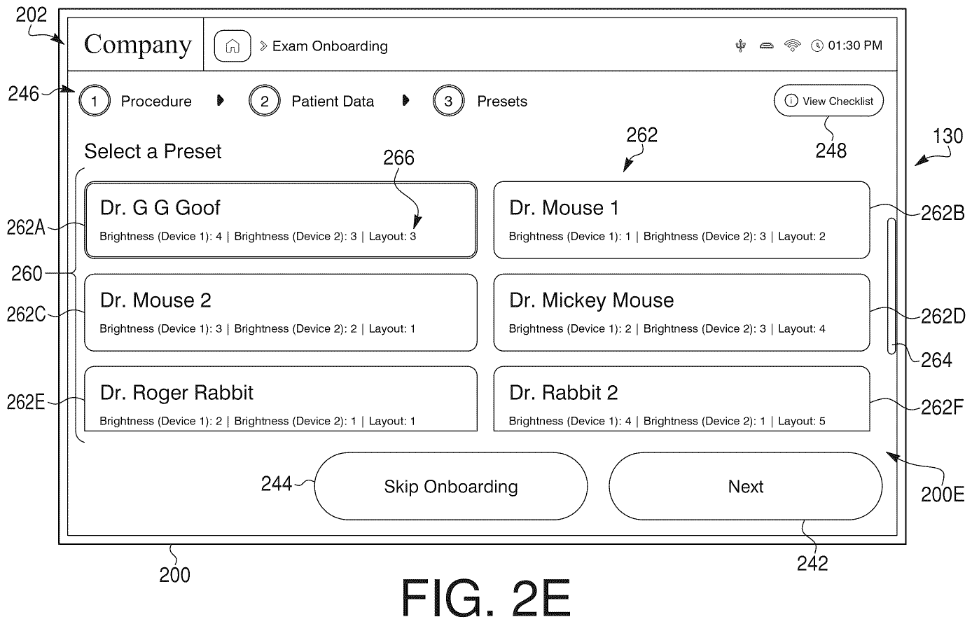

FIG. 1 depicts an exemplary medical system 100. Medical system 100 includes a medical device 102, a computing system 104, and one or more display device(s) 106. In some examples, medical system 100 may also include one or more optional server side system(s) 108 and an optional network 110 to communicatively couple at least computing system 104 to optional server side system(s) 108 and/or one or more additional displays (not shown). As discussed in detail below, computing system 104 includes a display 144 configured to display one or more user interfaces 130 (e.g., one or more graphical user interfaces (GUIs)) generated by computing system 104. In some examples, display 144 is an input/output (IO) device, such as a touch screen 200 (FIGS. 2A-2E, 3, 4A-4C, and 5). The user may manipulate user interface 130 (e.g., via touch screen(s) 200) to select and/or adjust the layout or orientation of one or more images or videos, for example, obtained from medical device 102, and/or other data or information on one or more display devices 106. In these aspects, the user may manipulate user interface 130 to define a layout via which images or videos from one or more imaging devices or other input sources connected to the computing system 104 can be simultaneously displayed. For example, computing system 104 may define the layout to display one or more images or videos that are being concurrently taken or recorded, and/or computing system 104 may define the layout to display one or more previously saved or previously taken images or videos (e.g., stored locally by computing system 104 and/or remotely in one of server side system(s) 108).

Medical device 102 may be used to perform a diagnostic and/or interventional medical procedure on a patient, for example, when connected to computing system 104. Medical device 102 may include a handle 112, a shaft 114, and an umbilicus 116. At least a portion of shaft 114, including a distal end 115 of shaft 114, may be inserted into and navigated through a body lumen of a patient to a target site during a medical procedure, such that distal end 115 is positioned proximate to the target site. Distal end 115 may include one or more illumination devices 118 and one or more imaging devices 120. Distal end 115 may also include distal openings for one or more of working channel(s) 122, suction channel(s) 124, and/or fluid or irrigation channel(s) 126.

Distal end 115 is shown in FIG. 1 as distal or front facing, with its components facing distally. However, in other examples, distal end 115, may be side-facing. That is, illumination devices 118, imaging device 120, and optionally one or more other distal end components, such as openings of one or more of working channel 122, suction channel 124, and/or fluid irrigation channel 126, may face radially outward, perpendicularly, approximately perpendicularly, or otherwise transverse to a longitudinal axis of shaft 114 and distal end 115. Additionally or alternatively, distal end 115 may include one or more imaging devices 120 that face in more than one direction. For example, a first imaging device 120 may face radially outward, and a second imaging device 120 may face distally (approximately parallel to a longitudinal axis of distal end 115 and shaft 114).

Medical device 102 may be a single use device that is discarded or disposed of upon disconnection from computing system 104 (e.g., at an end of the medical procedure). Therefore, although only one of medical device 102 is shown in FIG. 1, system 100 may include one or more other medical devices 102 that are the same or similar to medical device 102, and may also be removably connected to computing system 104 for performing medical procedures. Alternatively, one or more or portions of medical device 102 (e.g., handle 112, umbilicus 116, etc.) may be reusable, for example, in more than one medical procedure.

In some examples, and as shown in FIG. 1, medical device 102 may be an endoscope or other type of scope or medical device having imaging capabilities, such as a cholangioscope, bronchoscope, ureteroscope, duodenoscope, gastroscope, endoscopic ultrasonography (“EUS”) scope, colonoscope, laparoscope, arthroscope, cystoscope, aspiration scope, sheath, or catheter, among other examples.

Illumination devices 118 may be configured to receive and/or emit light to illuminate areas of the patient's body (e.g., the target site) during the medical procedure to facilitate imaging of the target site by imaging device 120. Illumination devices 118 may include one or more LEDs, incandescent light sources, optical fibers, and/or other illuminators. Imaging device 120 may be configured to continuously or intermittently capture image signals during the medical procedure as distal end 115 of medical device 102 is inserted into and navigated through the body lumen of the patient to the target site. Imaging device 120 may include one or more cameras, one or more image sensors (including analog and/or digital sensors), one or more endoscopic viewing elements, or one or more optical assemblies including one or more image sensors and one or more lenses, among other similar devices.

Working channel 122 may be a lumen that extends through shaft 114 and provides for delivery of instruments or treatment-related objects to the target site and/or removal of objects from the target site at distal end 115. Suction channel 124 may be a lumen that extends through shaft 114 and provides suctioning functionality at distal end 115. Fluid or irrigation channel 124 may be a lumen that extends through shaft 114 and provides irrigation functionality at distal end 115, for example, for cleaning imaging device 120, clearing the target site for better visualization, and/or otherwise delivering fluid to the target site.

A distal portion of shaft 114 that is connected to distal end 115 may have a steerable section. The steerable section may include, for example, one or more articulation joints (not shown). Shaft 114 and the steerable section may include a variety of structures that are known or may become known in the art.

Handle 112 may include one or more actuators. The actuators may provide control over one or more of the steerable section, an elevator (e.g., within a distal portion of working channel 122), and/or imaging functions at distal end 115 of shaft. Additionally, one or more actuators may also control or allow for the provision of air, water, suction, etc. For example, handle 112 may include control knobs 128, 129 for left, right, up, and/or down control of the steerable section of shaft 114. For example, one of control knobs 128, 129 may provide left/right control of the steerable section, and the other of control knobs 128, 129 may provide up/down control of the steerable section. Although not shown, handle 112 may further include one or more locking mechanisms (e.g., knobs or levers) for braking and/or preventing steering of the steerable section in at least one of an up, down, left, or right direction. Furthermore, although not shown, handle 112 may include an elevator control lever (e.g., if medical device 102 is a duodenoscope). The elevator control lever may raise and/or lower an elevator, via a connection between the lever and an actuating wire that extends from the lever, through shaft 114, to the elevator.

Additionally, medical device 102 may include one or more electronics. For example, medical device 102 may include one or more memory devices. The one or more memory devices may be configured to store calibration and/or manufacturing data, for example, including one or more identifiers, which may help to establish communication between medical device 102 and computing system 104 and/or set up operating parameters of medical device 102 upon connection to computing system 104. The identifier may include a serial number or other similar information for identifying medical device 102. In some examples, a portion of the identifier may indicate a general type of medical device 102, in addition to providing information unique to the particular medical device 102 of that general type. The calibration and manufacturing data may be written to the memory device(s) during manufacturing.

Umbilicus 116 connects handle 112, shaft 114, distal end 115, and components at distal end 115 to one or more sources of, for example, power, imaging device control, image processing, light, light control, and/or display equipment. As one example, umbilicus 116 may also connect to computing system 104 (e.g., via a connector plug 117), and support the transmission of various data signals between computing system 104 and medical device 102. For example, umbilicus 116 may support the transmission of identification data between medical device 102 and computing system 104 to facilitate usage monitoring. Additionally, umbilicus 116 may support the transmission of power and control signals from computing system 104 to medical device 102, and particularly to imaging device 120 and illumination devices 118. Further, umbilicus 116 may support the transmission of image data from imaging device 120 to computing system 104 for processing and display, for example, via display device(s) 106.

Computing system 104 may be a controller, a control unit, a computing device, or other similar standalone processing unit separate from and removably connectable to medical device 102. Computing system 104 may include a memory 140 and one or more processor(s) 142. Memory 140 may store instructions to be executed by processor(s) 142 to cause computing system 104 to perform corresponding operations. Memory 140 may also include one or more data stores. Additionally or alternatively, computing system 104 may include one or more data stores separate from memory 140. In some examples, processor(s) 142 may be or include a field-programmable gate array (FPGA), a digital signal processing (DSP) processor, a graphics processing unit (GPU), or the like.

In some aspects, processor(s) 142 may include at least one image and/or video processor configured to process, based on the stored instructions, image and/or video data captured by imaging components of medical device 102 and provided to computing system 104 to generate images and/or video.

As mentioned above, computing system 104 generates one or more user interfaces 130. The one or more user interfaces 130 may display various aspects of system 100 (e.g., details of medical device 102) and/or one or more procedures to the user(s) on display 144 (e.g., touch screen 200) and/or one or more other display devices 106. As discussed in detail below, the one or more user interfaces 130 may allow for one or more users to control (e.g., customize, adjusting, etc.) the display, layout, orientation, information, etc. presented on one or more display devices 106. For example, the user may manipulate the one or more user interfaces 130 to adjust a number of screens to be displayed on one or more display devices 106. In some aspects, the number of screens displayed on display device(s) 106 may be based on a number of image and/or video inputs, for example, from imaging device 120 and/or other imaging devices. The other imaging devices may be a part of medical device 102 (e.g., a side viewing imaging device) or may be separate from medical device 102 (e.g., imaging devices generating X-ray images, MRI images, CT images, fluoroscopy images, etc.). In some aspects, computing system 104 may be connected to multiple medical devices 102, and thus may receive image or video signals from each of the multiple medical devices 102. In these aspects, user interface 130 may allow the user to adjust the number, size, arrangement, layout, orientation, and/or other details of the images or videos from the multiple medical devices 102. In some aspects, computing system 104 may receive one or more inputs or selections causing computing system 104 to save one or more images and/or videos (e.g., in memory 140 and/or server side system(s) 108, as discussed below) to be displayed (e.g., on one or more display device(s) 106). In these aspects, computing system 104 may cause the saved one or more images and/or videos to be included in the display of the image or video signals from the medical device(s) 102.

Computing system 104 may further include an optional communication interface 146 for providing connectivity to optional network 110 to facilitate communication with optional server side system(s) 108. Although not shown in FIG. 1, optional communication interface 146 may also provide connectivity to medical device 102 and/or display device(s) 106. In some examples, a communicative connection between computing system 104 and medical device 102 and/or computing system 104 and display device(s) 106 may be at least partially supported via optional network 110.

Although only one computing system 104 is shown in FIG. 1, in some examples, system 100 may include one or more other computing systems 104 that are the same or similar to computing system 104 to which medical device 102 (or another medical device) may also be removably connected. To provide an illustrative example, within a medical facility, multiple procedural suites may each include one computing system 104 that remains located in that suite.

Display device(s) 106 may be configured to display data associated with one or more of medical device 102 and/or computing system 104. In some examples, displayed data may include images and/or videos captured by medical device 102 and/or one or more other medical devices or input sources connected to computing system 104. As mentioned, in some examples, displayed data may include one or more saved images and/or videos (e.g., received from local storage in memory 140 of computing system 104 and/or from remote storage in one of optional server side system(s) 108). The images, videos, and/or other data may be displayed in accordance with a user-adjusted or customized layout (e.g., the layout defined via interactions with user interface 130). Display device(s) 106 may include one or more a combination of monitors, computing device screens, touch screen display devices, etc. In some examples, one or more of display device(s) 106 may be a separate or external device from computing system 104 that is communicatively coupleable to computing system 104 via wired and/or wireless connections. In other examples, at least one of display device(s) 106 may be a display or screen of computing system 104 itself (e.g., separate from display 144 and/or the same display 144).

In some examples, computing system 104 may generate, or may cause to be generated, one or more graphical user interfaces, including user interface 130, based on instructions or information stored in memory 140, instructions or information received from one or more optional server side system(s) 108, and/or the like and may cause the graphical user interfaces to be displayed via display 144 and/or display device(s) 106. The graphical user interfaces may include text, visual elements, controls, and/or the like, in addition to the displayed data. Display 144 and/or display device(s) 106 may include a touch screen or a display with other input systems (e.g., a mouse, keyboard, voice, etc.) for an operator of computing system 104 to control functions of computing system 104, medical device 102 via computing system 104, and/or display device(s) 106.

One or more components of system 100, such as medical device 102, computing system 104, and/or display device(s) 106, may be capable of network connectivity, and may communicate with one another over a wired network or a wireless network, such as optional network 110. The network may be an electronic network. The network may include a wide area network (“WAN”), a local area network (“LAN”), personal area network (“PAN”), a cellular network (e.g., a 3G network, a 4G network, a 5G network, etc.), or the like. In other examples, the components of system 100 may communicate and/or connect to the network over universal serial bus (USB) or other similar local, low latency connections or direct wireless protocol. Components of system 100 may be connected via the network, using one or more standard communication protocols, such that the component may transmit and receive communications from each other across the network.

In some examples, when one or more of the components of medical system 100 are capable of connecting to optional network 110, such as at least computing system 104, medical system 100 may also include optional server side system(s) 108. Optional server side system(s) 108 may include one or more remote data storage systems for storing data generated by computing system 104 (e.g., image data). Additionally or alternatively, when medical device 102 includes an imaging system or device, optional server side system(s) 108 may include remote image processing systems. The remote image processing systems may be configured to perform at least a portion of the image processing, including but not limited, more resource intensive processes, such as machine learning processes (e.g., to conserve local resources of computing system 104 when network connectivity is available).

Although various components in medical system 100 are depicted as separate components in FIG. 1, it should be understood that a component or portion of a component in system 100 may, in some embodiments, be integrated with or incorporated into one or more other components. For example, one of display device(s) 106 may be integrated with computing system 104. In some embodiments, operations or aspects of one or more of the components discussed above may be distributed amongst one or more other components. Any suitable arrangement and/or integration of the various systems and devices of medical system 100 may be used.

As discussed in detail herein, various aspects of this disclosure may allow a user to customize or otherwise adjust the display of one or more images, videos, patient data, and/or other information before, during, or after a procedure. For example, user interface 130 (e.g., via touch screen 200) may receive one or more user inputs to select and manipulate the display of one or more images, videos, patient data, and/or other information. The specific examples included throughout this disclosure implement an endoscopic imaging system. However, it should be understood that techniques according to this disclosure may be adapted to other medical systems having any type of medical device with imaging capabilities that is connected to a computing system to enable operation thereof (e.g., to enable processing and display of image and/or video data). It should also be understood that the examples above are illustrative only. The techniques and technologies of this disclosure may be adapted to any suitable activity.

FIGS. 2A-2E illustrate various screens of user interface 130 displayed on touch screen 200 (e.g., one type of exemplary display 144). Each screen is a configuration of user interface 130 that includes a plurality of media indication and/or control elements (e.g., icons, buttons, etc.) to display various information or data and/or to allow for user selection and/or adjustment of various aspects of system 100. Specifically, FIG. 2A illustrates a home, pre-procedure, or pre-exam configuration (“home screen 200A”) of user interface 130 displayed on touch screen 200. As shown, home screen 200A may include an information bar 202, for example, at a top of home screen 200A. Information bar 202 may include a product and/or brand identifier 204 (e.g., Company). Information bar 202 may also include one or more connectivity indicators, for example, a first connection (e.g., USB 2.0) indicator 206, a second connection (e.g., USB-C or micro-USB) indicator 208, and a third connection (e.g., Wi-Fi or Ethernet) indicator 210. In some aspects, information bar 202 may include one or more other connection indicators, for example, a Bluetooth®, charging connection, etc. The one or more connectivity indicators may illuminate, flash, change color, or otherwise visually indicate when a connection is active, inactive, in an error state, etc. Moreover, information bar 202 may also include one or more time indicators, for example, a clock 212. In some aspects, home screen 200A may also include a version indicator 214 (e.g., Version 1.02), for example, indicative of the version of software being run by computing system 104. Furthermore, in some aspects, home screen 200A may include one or more additional product and/or brand identifiers 204A (e.g., Company), for example, positioned in a central portion of home screen 200A.

Home screen 200A may also include a mode selection portion or bar 216. Mode selection bar 216 may include a plurality of icons, and selection of one or more of the icons may cause computing system 104 to change from home screen 200A of user interface 130 to one or more other screens of user interface 130 displayed on touch screen 200. For example, home screen 200A may include a procedures icon 218, a start exam mode icon 220, and a settings icon 222. As discussed in greater detail below, selection of procedures icon 218 may cause computing system 104 to display a listing of one or more procedures (e.g., diagnostic procedures, treatment procedures, etc.) on touch screen 200 for viewing, selection, etc. Selection of start exam mode icon 220 may cause computing system 104 to display one or more screens of user interface 130 on touch screen 200 that facilitate a set up or initiation of an exam (e.g., a medical procedure) to be performed using one or more medical devices (e.g., medical device 102) coupled to computing system 104. One example screen that may be displayed upon selection of start exam mode icon 220 is a procedure selection configuration 200B of user interface 130, shown and described with reference to FIG. 2B. Selection of settings icon 222 may cause computing system 104 to display a listing of one or more settings (e.g., for medical device 102, computing system 104, display device 106, etc.) on touch screen 200, for example, for the user to set, adjust, modify, or otherwise view the one or more settings.

FIG. 2B illustrates procedure selection configuration 200B of user interface 130 displayed on touch screen 200. As shown, procedure selection configuration 200B may include information bar 202. Information bar 202 in procedure selection configuration 200B may also include one or more procedure stage indicators 230, such as “exam onboarding”, indicating that computing system 104, user interface 130, and touch screen 200 are in an examination onboarding stage. In some aspects, information bar 202 may include a return home icon 232. Selection of return home icon 232 may cause computing system 104, user interface 130, and touch screen 200 to return to a home mode (e.g., home screen 200A).

Additionally, procedure selection configuration 200B may include a procedure selection bar 234 including one or more selectable icons representing various types of procedures. For example, procedure selection bar 234 may include an ERCP procedure icon 236, an ERCP + Secondary Device procedure icon 238, and an Other icon 240. Selection of one of these icons may then display another configuration of user interface 130 on touch screen 200 that is relevant for the selected procedure. For example, selection of ERCP procedure icon 236 may display a configuration of user interface 130 for an endoscopic retrograde cholangiopancreatography (ERCP) procedure, where an endoscope is used to remove one or more gallstones, or otherwise treat or view aspects of a patient's liver, gallbladder, bile duct(s), or pancreas. Selection of ERCP + Secondary Device procedure icon 238 may then display a configuration of user interface 130 on touch screen 200 for an ERCP procedure performed with an endoscope and a secondary medical device (e.g., Boston Scientific SpyGlass™ DS II Direct Visualization System). In some aspects, selection of the other icon 240 may cause touch screen 200 to display a listing of one or more other or additional procedures, medical devices, etc. for user selection.

In some aspects, selection of one of these icons may cause touch screen 200 to display one or more trainings, onboarding programs, training videos, or other information via user interface 130. For example, the user may select one or more of the procedure icons (e.g., to highlight or illuminate the selected icon), and may then select a next icon 242, which may cause computing system 104, user interface 130, and touch screen 200 to display one or more trainings, onboarding programs, training videos, or other information for the selected procedure. For example, selecting next icon 242 may cause computing system 104 to display a search bar to search for one or more patients (e.g., saved or previously entered patient data or other information) on touch screen 200 for viewing, selection, etc. One example, screen that may be displayed upon selection of next icon 242 is a patient search configuration 200C of user interface 130, shown and described with reference to FIG. 2C. Alternatively, the user may select a skip onboarding icon 244 via touch screen 200 that is included in procedure selection configuration 200B, to proceed directly to a procedure mode, for example, bypassing the trainings, onboarding programs, training videos, etc.

In some aspects, procedure selection configuration 200B may include a status bar 246, for example, below information bar 202 and above procedure selection bar 234. Status bar 246 may illuminate, flash, highlight, or otherwise indicate a status associated with a procedure or pre-procedure process or stage (e.g., a status or current progression associated with the exam onboarding stage). For example, as shown in FIG. 2B, status bar 246 may include a procedure selection stage icon 246A, a patient data stage icon 246B, and a presets stage icon 246C. Furthermore, procedure selection configuration 200B may include a view checklist icon 248, where selection of view checklist icon 248 may cause computing system 104 to display one or more checklists or a listing of steps or tasks for one or more procedures via user interface 130 on touch screen 200. In some aspects, the one or more procedures for which one or more checklists may be displayed may correspond to or be a subset of the procedures that are listed in procedure selection bar 234.

FIG. 2C illustrates a patient search configuration 200C of user interface 130 on touch screen 200. Patient search configuration 200C may include at least some similar elements to procedure selection configuration 200B. For example, patient search configuration 200C may include one or more of information bar 202, next icon 242, skip onboarding icon 244, status bar 246, and view checklist icon 248, as discussed in detail above. As shown in FIG. 2C, status bar 246 may include both procedure selection stage icon 246A and patient data stage icon 246B illuminated or otherwise highlighted. Additionally, patient search configuration 200C may include a search bar 250, for example, for the user to search and/or add a patient that will be the subject of the selected procedure. The user may select search bar 250 to search for a patient, for example, via a patient identification (ID) number or name. Alternatively, the user may search for the patient by birth date or through other patient information. In these aspects, although not shown, user interface 130 may display a keyboard (e.g., including letters and/or numbers) for the user to type or input the patient ID number, name, or other information, for example, to search for the patient's information. In some examples, selecting next icon 242 may cause computing system 104 to display a listing of one or more patients (e.g., saved or previously entered patient data or other information) on touch screen 200 for viewing, selection, etc. In one example, a screen that may be displayed upon selection of next icon 242 is a patient selection configuration 200D of user interface 130, shown and described with reference to FIG. 2D.

FIG. 2D illustrates a patient selection configuration 200D of user interface 130 on touch screen 200. Patient selection configuration 200D may include at least some similar elements to procedure selection configuration 200B and/or patient search configuration 200C. For example, patient selection configuration 200D may include one or more of information bar 202, next icon 242, skip onboarding icon 244, status bar 246, and view checklist icon 248, as discussed in detail above. Additionally, as shown, search bar 250 may receive one or more inputs concerning patient information to search for the patient's information. For example, the user may enter the patient's identification number 252 (e.g., ENDO_6855155), or a portion of the patient's identification number to search for the patient's information. As a result, patient selection configuration 200D may display one or more patient results 254 for the user to select (e.g., touch or otherwise select via touch screen 200). Once a patient result is selected, from patient results 254, patient selection configuration 200D may highlight, illuminate, or otherwise indicate that the patient result has been selected. The user may then select next icon 242 to move to the next step(s) of the pre-procedure or onboarding process. In some examples, selecting next icon 242 may cause computing system 104 to display a listing of one or more preset configurations (e.g., saved or previously entered device settings) on touch screen 200 for viewing, selection, etc. In one example, a screen that may be displayed upon selection of next icon 242 is a preset selection configuration 200E of user interface 130, shown and described with reference to FIG. 2E.

FIG. 2E illustrates a preset selection configuration 200E of user interface 130 on touch screen 200. Preset selection configuration 200E may include at least some similar elements to procedure selection configuration 200B, patient search configuration 200C, and patient selection configuration 200D. For example, preset selection configuration 200E may include one or more of information bar 202, next icon 242, skip onboarding icon 244, status bar 246, and view checklist icon 248, as discussed in detail above. As mentioned above, status bar 246 may illuminate, flash, highlight, or otherwise indicate a status associated with a procedure or pre-procedure process or stage (e.g., a status or current progression associated with the exam onboarding stage). For example, as shown in FIG. 2E, status bar 246 may include procedure selection stage icon 246A, patient data stage icon 246B, and presets stage icon 246C, with each of icons 246A, 246B, and 246C illuminated, flashing, highlighted, etc. Additionally, as shown, preset selection configuration 200E includes a preset listing 260, for example, including a plurality of preset configurations or results 262. Preset listing 260 may include all of the preset configurations for medical system 100. Alternatively, preset listing 260 may include only a subset of the preset configurations for medical system 100, for example, including only the preset configurations for a selected procedure (e.g., selected via procedure selection configuration 200B (FIG. 2B)).

In any of these aspects, preset results 262 may include a plurality of preset results 262A, 262B, 262C, 262D, 262E, 262F, etc. Each preset result may include a preset identifier, for example, the user's name and/or a numerical indication. For example, if a user (e.g., Dr. Mouse) has stored more than one preset configuration, then preset listing may include more than one preset result with Dr. Mouse's name, along with numerical differences (e.g., Dr. Mouse 1 in one preset result 262B, Dr. Mouse 2 in another preset result 262C, and Dr. Mickey Mouse in yet another preset result 262D). Furthermore, if the number of preset results 262 exceeds the space available on preset listing 260 of preset selection configuration 200E of user interface 130, then preset selection configuration 200E may include a scroll bar 264, for example, selectively movable to scroll or toggle through the preset results 262. Furthermore, as shown, each preset result 262 may include one or more preset preferences 266. For example, each preset result 262 may include a desired brightness for one or more medical devices and/or a desired layout, as discussed below. For example, each preset result 262 may include a desired brightness (e.g., for one or more illumination devices 118) for a first medical device (e.g., Exalt Model D) and also a desired brightness for a second medical device (e.g., Boston Scientific SpyGlass™ DS II Direct Visualization System). Each preset result 262 may include a desired layout of one or more image, video, data, and/or other information displays. Although not shown, preset selection configuration 200E may include a search bar, for example, for a user to search for a desired preset result.

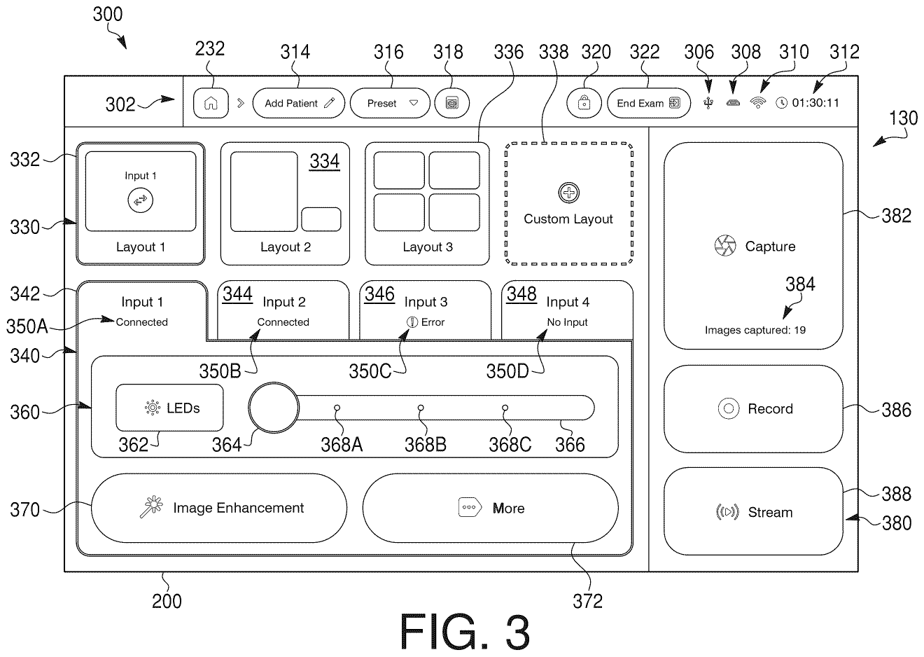

FIG. 3 illustrates another example screen (e.g., an active procedure screen 300) of user interface 130 that may be displayed on touch screen 200 of computing system 104. Active procedure screen 300 includes a plurality of media indication and/or control elements (e.g., icons, buttons, etc.) to display various information or data and/or to allow for user selection and/or adjustment of various aspects of system 100 (FIG. 1).

As shown, active procedure screen 300 includes a task or information bar 302, for example, at a top portion of active procedure screen 300. Information bar 302 may be similar to information bar 202. For example, information bar 302 may include one or more of a first connection indicator 306, a second connection indicator 308, a third connection indicator 310, and/or a clock 312. In some aspects, information bar 302 may include one or more other connection indicators, timers, or other icons or information. Although not shown, information bar 302 may include a brand identifier, as discussed above. Additionally, information bar 302 may include a return home icon 232 to return to a home screen (e.g., home screen 200A), as discussed above.

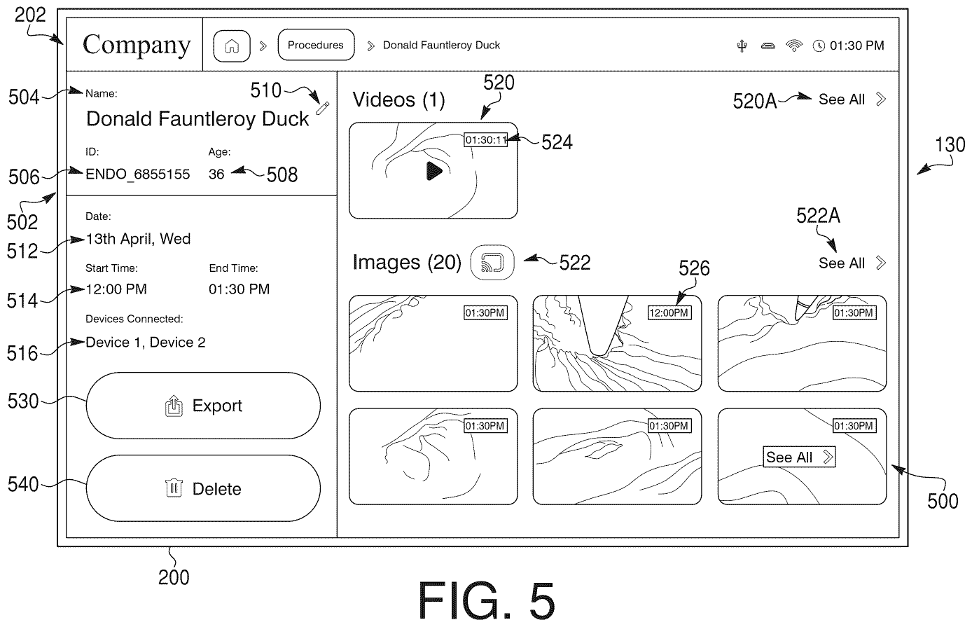

Information bar 302 may also include an add patient icon 314, for example, selectable to add a patient or select patient information (e.g., from a memory and/or as discussed with respect to patient search configuration 200C above). Information bar 302 may include a preset selection icon 316, for example, selectable to display a preset selection listing (e.g., in a drop down menu) and/or a preset selection listing (e.g., preset listing 260 as discussed with respect to preset selection configuration 200E above). Furthermore, information bar 302 may include an image and/or video review icon 318. Image and/or video review icon 318 may be selectable to transition user interface 130 to an image and/or video review display (e.g., FIG. 5). Selection of image and/or video review icon 318 may also end any active procedure, initiate a display of a procedure summary, and/or return to procedure selection screen, for example, to perform another procedure. In these aspects, selection of image and/or video review icon 318 may allow the user to view images and/or video (or other data or information) from previous and/or completed procedures.

Information bar 302 may include a lock icon 320, for example, selectable to lock user interface 130. In this aspect, locking user interface 130 may prevent any received inputs from being processed to help to ensure that the user's selections are not accidentally adjusted (e.g., via inadvertent contact with touch screen 200) or otherwise interfered with. For example, the user may lock user interface 130 by selecting lock icon 320 (e.g., touching lock icon 320 for a period of time). Once user interface 130 is locked, lock icon 320 may transition to an unlock icon (not shown). Then, the user may unlock user interface 130 by selecting the unlock icon (e.g., touching the unlock icon for a period of time).

Information bar 302 may further include an end examination icon 322. For example, selecting end examination icon 322 may end any on-going or active procedure. In some aspects, selecting end examination icon 322 may cause touch screen 200 to display, via user interface 130, a confirmatory question for the user (e.g., “Are you sure you want to end the examination?”), which may help to ensure that the user does not inadvertently end a procedure. Alternatively or additionally, in some aspects, the user may have to select (e.g., touch) end examination icon 322 for a period of time before the procedure is ended (e.g., by exiting active procedure screen 300). Ending the procedure may return user interface 130 to home screen 200A (FIG. 2A) or may transition user interface 130 to an end or summary screen (e.g., FIG. 5).

Moreover, active procedure screen 300 includes a layout selection bar 330. Layout selection bar 330 may include a plurality of pre-set or predefined layouts for the display of one or more image or video signals. Layout selection bar 330 may include a first layout icon 332 (“layout 1” in FIG. 3), for example, for displaying one set of image or video signals associated with one input source. Layout selection bar 330 may include a second layout icon 334 (“layout 2” in FIG. 3), for example, for displaying two sets of image or video signals associated with two input sources. Layout selection bar 330 may include a third layout icon 336 (“layout 3” in FIG. 3), for example, for displaying four sets of image or video signals associated with four input sources. Layout icons 332, 334, and 336 may include visual indicators of the layout or arrangement of a number of tiles representing the respective number of sets of image or video signals. Selecting one of layout icons 332, 334, and 336 may allow the user to select a pre-defined arrangement of the one or more sets of image or video signals for the procedure. Furthermore, and as discussed in greater detail below, layout selection bar 330 includes a custom layout icon 338 (“custom layout” in FIG. 3). Selecting custom layout icon 338 may allow for a user to select or customize arrangement of one or more sets of image or video signals within a customized layout for a procedure. Additionally, the user may save the customized layout for use in the current procedure and/or for use in a future procedure.

Additionally, active procedure screen 300 may include one or more input tabs, for example, arranged in an input tab section 340 of user interface. Input tab section 340 may include a tab for each of one or more inputs, for example, inputs (i.e., physically wired and/or wireless inputs) of computing system 104. In some aspects, computing system 104 may include four inputs, and input tab section 340 may include a first input tab 342, a second input tab 344, a third input tab 346, and a fourth input tab 348. Nevertheless, input tab section 340 of active procedure screen 300 may include fewer or more than four input tabs.

Input tabs 342, 344, 346, and 348 may include one or more status indicators. For example, each input tab 342, 344, 346, and 348 may include a respective status indicator 350A, 350B, 350C, and 350D. The status indicator may indicate a status of the respective input (e.g., connector plug 117 being coupled to computing system 104 to connect medical device 102 to computing system 104 in FIG. 1). For example, the status indicator may indicate that the input is connected (i.e., status indicators 350A and 350B for input tabs 342 and 344) to a respective medical device (e.g., medical device 102). In another example, the status indicator may indicate that a given input is experiencing an error (i.e., status indicator 350C for input tab 346), for example, due to an improper or faulty connection to the respective medical device. In some aspects, the error indication may be indicative of the connected device having been used too many times and/or too long ago (e.g., a usage or reuse issue). Furthermore, in yet another example, the status indicator may indicate that no device is connected to a given input (i.e., status indicator 350D for input tab 348).

One or more of input tabs 342, 344, 346, and 348 may also include one or more indications and/or controls. In some aspects, the indications and/or controls may be device specific, for example, depending on the type of medical device connected to the respective input. Alternatively, the indications and/or controls may be generally applicable to all medical devices connected to the inputs.

One or more input tabs 342, 344, 346, and 348 may include an illumination control section 360. Illumination control section 360 may include an on/off icon 362 that is selectively activated or deactivated to toggle one or more illumination devices (e.g., illumination device 118) between an on configuration and an off configuration. Additionally, in some aspects, selecting (i.e., touching) on/off icon 362 for different amounts of time and/or different numbers of touches may increase and/or decrease a brightness of one or more illumination devices. Furthermore, illumination control section 360 may include one or more illumination controls or sliders 364. For example, slider 364 may be movable (e.g., longitudinally or linearly movable based on touching and/or sliding) along a track 366. Additionally, track 366 may include one or more incremental markers, for example, a first marker 368A, a second marker 368B, and a third marker 368C. For example, markers 368A, 368B, and 368C may be evenly or unevenly spaced along a length of track 366 and may help to indicate a position of slider 364 that corresponds to a brightness of the one or more illumination devices. For example, slider 364 being positioned at or about first marker 368A may correspond to approximately 25% brightness of illumination device(s), and slider being positioned at or about second marker 368B may correspond to approximately 50% brightness of illumination devices. Furthermore, slider 364 being positioned at or about third marker 368C may correspond to approximately 75% brightness, and slider 364 being positioned at or about an end (e.g., a right end in FIG. 3) of track 366 may correspond to approximately 100% brightness.

In some aspects, one or more input tabs 342, 344, 346, and 348 may include an image enhancement icon 370. For example, a user may select image enhancement icon 370 to incorporate one or more image enhancement algorithms and/or artificial intelligence processing and/or modification in the display of the image and/or video data from the corresponding medical device. For example, toggling on image enhancement icon 370 may cause computing system 104 to modify or otherwise adjust the image and/or video data from the corresponding medical device for the selected input. Additionally, one or more input tabs 342, 344, 346, and 348 may include a more icon 372. For example, selecting more icon 372 may cause user interface 130 to display one or more additional options, settings, and/or information for the corresponding medical device. In some aspects, selecting more icon 372 may cause user interface 130 to display one or more image adjustment parameters to correspond to user preferences, for example, including the adjustment of one or more of color, contrast, and/or sharpness. Additionally or alternatively, selecting more icon 372 may cause user interface to display one or more device settings screens, for example, to display and/or adjust one or more device settings (e.g., of one or more medical devices 102, computing system 104, display device(s) 106, etc.).

In some aspects, one or more of input tabs 342, 344, 346, 348 may indicate a type of device that is connected to the respective input. For example, although not shown, one or more of input tabs 342, 344, 346, 348 may include an indication of the type of device that is coupled to the respective input of computing system 104.

Furthermore, active procedure screen 300 may include one or more image and/or video control sections 380. For example, as shown in FIG. 3, image and/or video control section 380 may include an image capture icon 382, for example, selectable to cause computing system 104 to signal one or more imaging devices (e.g., imaging device 120) to capture one or more still images for storage (e.g., from the continuous feed of live images being captured by imaging device 120). Additionally, image and/or video control section 380 may include a counter and/or an indication 384 of a total number of still images captured in the procedure and/or for the respective medical device. In some aspects, image and/or video control section 380 may include a record icon 386. For example, selectively toggling on record icon 386 may cause computing system 104 to signal the respective medical device to record video for storage. Moreover, image and/or video control section 380 may include a stream icon 388. For example, selectively toggling on stream icon 388 may cause computing system 104 to signal computing system 104 to stream (e.g., transmit data in real-time) to one or more additional displays, broadcast signals, etc. (e.g., via the Internet and/or network 110). In these aspects, image and/or video control section 380 may allow the user to perform image capture, recording, and/or streaming functions with relation to one or more of input tabs 342, 344, 346, 348. For example, image and/or video control section 380 may allow the user to perform image capture, recording, and/or streaming functions with relation to only a highlighted or selected input tab. Alternatively, image and/or video control section 380 may allow the user to perform image capture, recording, and/or streaming functions with relation to one or more inputs that are being displayed on display devices 106.

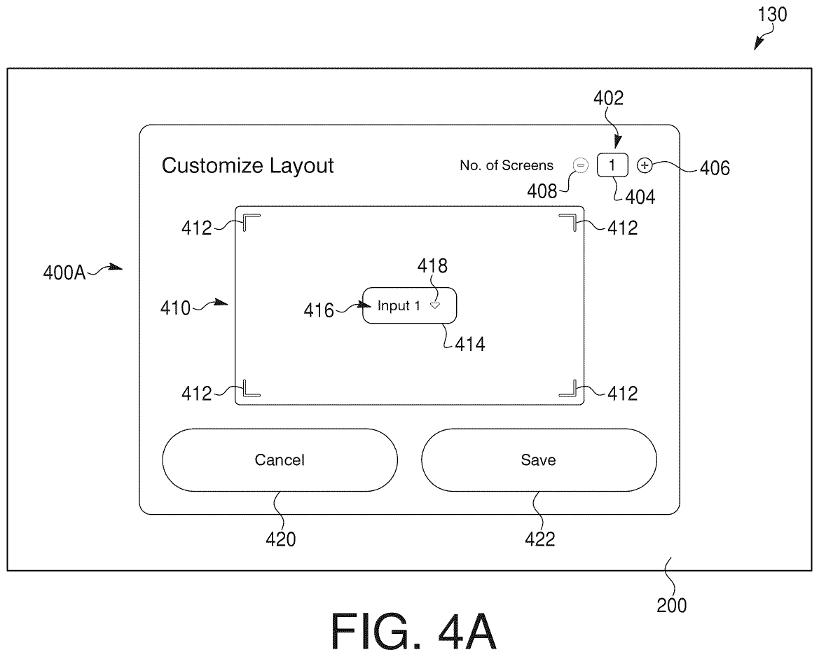

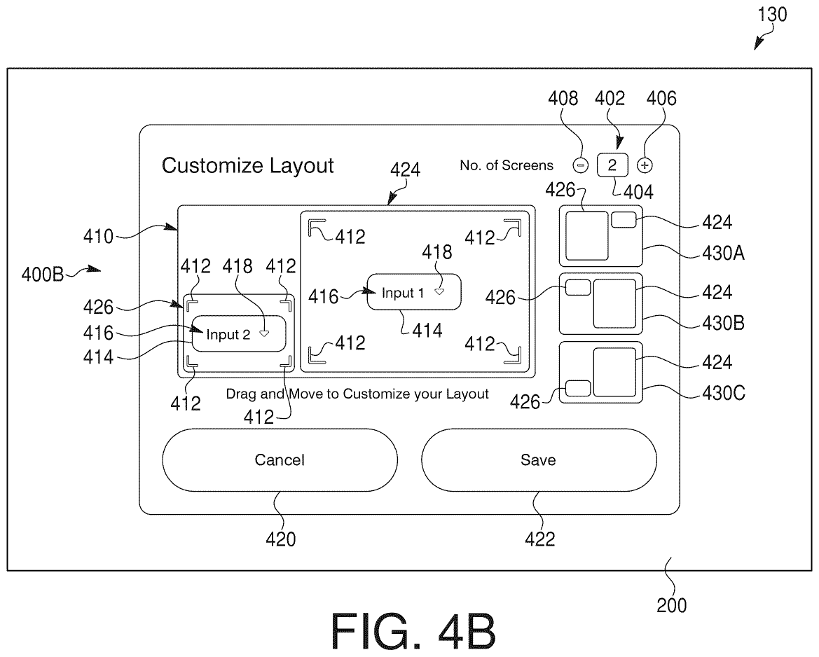

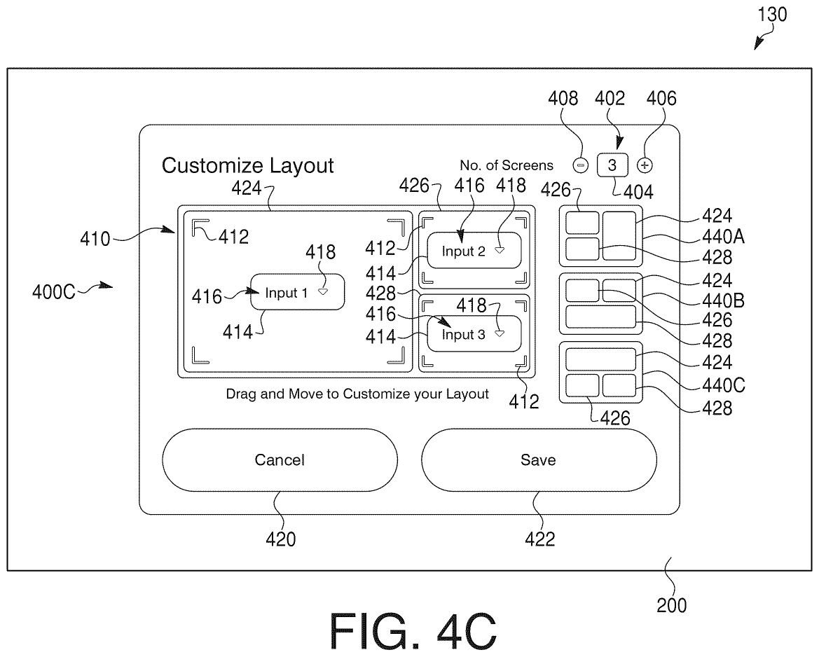

FIGS. 4A-4C illustrate various exemplary customize layout screens. For example, as mentioned above, the user(s) may select to customize a layout screen by selecting the “custom layout” media control element or icon 338 on active procedure screen 300. Additionally, the user may toggle or otherwise selectively move between the exemplary customize layout screens of FIGS. 4A-4C. Although not shown, the customize layout screens of FIGS. 4A-4C may be overlaid over one or more of the screens, displays, or configurations of user interface 130 discussed herein, such as active procedure screen 300.

FIG. 4A illustrates a first exemplary customize layout screen 400A of user interface 130 displayed on touch screen 200. For example, the user may select (touch, depress, etc.) custom layout icon 338, as shown in FIG. 3, and computing system 104 may cause first customize layout screen 400A to be displayed on user interface 130. As shown, first customize layout screen 400A may include a number of screen(s) control portion 402, for example, in a top right corner portion. Number of screen(s) control portion 402 may include a display icon 404 (e.g., indicating the number of screens that is currently selected), an add screen icon 406 (e.g., shown as a plus sign in FIG. 4A), and a reduce screen icon 408 (e.g., shown as a minus sign in FIG. 4A).

Furthermore, first customize layout screen 400A may include a frame indication icon 410, for example, indicative of the size, shape, and/or position of the image and/or video information (e.g., a tile size) to be displayed. Frame indication icon 410 may include a plurality of frame edges or corners 412, for example, L-shaped corners. The user may manipulate corners 412 (e.g., via touching, sliding, pinching, or otherwise manipulating touch screen 200) to adjust a size, shape, and/or position of frame indication icon 410. For example, moving one corner 412 outward (i.e., away from the other corners 412) may enlarge the size of frame indication icon 410, and moving one corner 412 inward (i.e., toward the other corners 412) may reduce the size of frame indication icon 410. Additionally, the user may manipulate frame indication icon 410 (e.g., portions of frame indication icon 410 that are not corners 412) to move the position of frame indication icon 410 on first customize layout screen 400A (e.g., drag and drop).

First customize layout screen 400A may also include an input selection icon 414, for example, positioned on a portion (e.g., a middle portion) of frame indication icon 410. Input selection icon 414 may include an indication 416 of the currently selected input (e.g., input 1, as shown in FIG. 4A). Additionally, input selection icon 414 may also include a selection change icon 418. For example, selecting selection change icon 418 may cause computing system 104 to display a drop down menu of the inputs (e.g., input 1, input 2, input 3, and input 4). In this aspect, the user may select a different input, such that the image and/or video data from the selected input is displayed, for example, on display device 106 in accordance with the size, shape, and/or position of the image and/or video information (e.g., the tile size) indicated by the frame indication icon 410. In some aspects, the drop down menu may only display connected inputs, for example, not showing input(s) with errors and/or no device being connected to the input(s) (e.g., with error as the status indicator 350C and/or with no input as the status indicator 350D).

Additionally, first customize layout screen 400A may also include one or more of a cancel icon 420 and/or a save icon 422. For example, selection of cancel icon 420 may cause computing system 104 to reset the size, shape, and/or position of frame indication icon 410 and corners 412 to a default size, shape, and/or position. Additionally, selection of save icon 422 may cause computing system 104 to save the size, shape, and/or position of frame indication icon 410 and corners 412. In this aspect, computing system 104 may save the selected size, shape, and/or position of frame indication icon 410, and may display the selected size, shape, and/or position as a preset result 262 in preset listing 260 (FIG. 2E). In some aspects, computing system 104 may allow for the user to customize the layout during a procedure, and also allow for the user to return to the customized layout in a future procedure.

FIG. 4B illustrates a second exemplary customize layout screen 400B of user interface 130 displayed on touch screen 200. For example, the user may select (touch, depress, etc.) custom layout icon 338, as shown in FIG. 3, and computing system 104 may cause second customize layout screen 400B to be displayed on user interface 130 (e.g., if two inputs to computing system 104 are detected). Alternatively or additionally, the user may add a screen (e.g., by touching, depressing, etc.) add screen icon 406 when on first customize layout screen 400A, and computing system 104 may cause second customize layout screen 400B to be displayed. As shown, second customize layout screen 400B may include number of screen(s) control portion 402, including display icon 404, add screen icon 406, and reduce screen icon 408, as discussed above. The number of available screens may be based on the number of medical devices connected to computing system 104.

Furthermore, second customize layout screen 400B may include frame indication icon 410, for example, indicative of the size, shape, and/or position of the image and/or video information (e.g., a tile size) to be displayed. Frame indication icon 410 may include two image or video display tiles, for example, to display two screens. For example, a first image or video display tile 424 may correspond to a size, shape, and/or position of the image or video to be displayed from a first input (e.g., input 1), and a second image or video display tile 426 may correspond to a size, shape, and/or position of the image or video to be displayed from a second input (e.g., input 2). Each of image or video display tiles 424 and 426 may include a plurality of frame edges or corners 412, for example, L-shaped corners, and the user may manipulate corners 412 (e.g., via touching, sliding, pinching, or otherwise manipulating touch screen 200) to adjust a size, shape, and/or position of each of first and second image or video display tiles 424, 426, as discussed above. Additionally, the user may manipulate first and second image or video display tiles 424, 426 (e.g., portions of first and second image or video display tiles 424, 426 that are not corners 412) to move the position of first and second image or video display tiles 424, 426 on second customize layout screen 400B (e.g., drag and drop).

In some aspects, second customize layout screen 400B may include one or more preset or saved layouts or configurations, for example, to the right of frame indication icon 410 in FIG. 4B. For example, second customize layout screen 400B may include a first saved layout 430A, a second saved layout 430B, and a third saved layout 430C. The saved layouts may differ in the size and/or position of first and second image or video display tiles 424, 426. The user may select (e.g., touch, depress, etc.) one of the saved layouts to transition frame indication icon 410 to a layout corresponding to the selected layout, which the user may then modify (e.g., via corners 412) if desired.

Second customize layout screen 400B may also include input selection icon 414, for example, positioned on a portion (e.g., a middle portion) of each of first and second image or video display tiles 424, 426. As discussed above, each input selection icon 414 may include indication 416 of the currently selected input (e.g., input 1 for first image or video display tile 424 and input 2 for second image or video display tile 426, as shown in FIG. 4B). In some aspects, computing system 104 may only display available inputs, that is, when medical devices are properly connected to the respective inputs on computing system 104. Additionally, input selection icons 414 may also include selection change icon 418, as discussed above. Second customize layout screen 400B may also include one or more of cancel icon 420 and/or save icon 422, as discussed above. Saving a customized layout may cause computing system 104 to display the saved layout as a saved layout at a later time (e.g., instead of or in addition to one of saved layouts 430A, 430B, 430C).

FIG. 4C illustrates a third exemplary customize layout screen 400C of user interface 130 on touch screen 200. For example, the user may select (touch, depress, etc.) custom layout icon 338, as shown in FIG. 3, and computing system 104 may cause third customize layout screen 400C to be displayed on user interface 130 (e.g., if three inputs to computing system 104 are detected). Alternatively or additionally, the user may add a screen (e.g., by touching, depressing, etc.) add screen icon 406 when on second customize layout screen 400B, and computing system 104 may cause third customize layout screen 400C to be displayed. As shown, third customize layout screen 400C may include number of screen(s) control portion 402, including display icon 404, add screen icon 406, and reduce screen icon 408, as discussed above.

Furthermore, third customize layout screen 400C may include frame indication icon 410, for example, indicative of the size, shape, and/or position of the image and/or video information (e.g., a tile size) to be displayed. Frame indication icon 410 may include three image or video display tiles, for example, to display three screens. For example, first image or video display tile 424 may correspond to a size, shape, and/or position of the image or video to be displayed from a first input (e.g., input 1). Additionally, second image or video display tile 426 may correspond to a size, shape, and/or position of the image or video to be displayed from a second input (e.g., input 2), and a third image or video display tile 428 may correspond to a size, shape, and/or position of the image or video to be displayed from a third input (e.g., input 3). Each of image or video display tiles 424, 426, 428 may include a plurality of frame edges or corners 412, for example, L-shaped corners, and the user may manipulate corners 412 (e.g., via touching, sliding, pinching, or otherwise manipulating touch screen 200) to adjust a size, shape, and/or position of each of first, second, and/or third image or video display tiles 424, 426, 428, as discussed above. Additionally, the user may manipulate first, second, and/or third image or video display tiles 424, 426, 428 (e.g., portions of first and second image or video display tiles 424, 426, 428 that are not corners 412) to move the position of first, second, and/or third image or video display tiles 424, 426, 428 on third customize layout screen 400C (e.g., drag and drop).

Additionally, in some aspects, third customize layout screen 400C may include one or more preset or saved layouts or configurations, for example, to the right of frame indication icon 410 in FIG. 4C. For example, third customize layout screen 400C may include a first saved layout 440A, a second saved layout 440B, and a third saved layout 440C. The saved layouts may differ in the size, shape, and/or position of first, second, and third image or video display tiles 424, 426, 428. The user may select (e.g., touch, depress, etc.) one of the saved layouts to transition frame indication icon 410 to a layout corresponding to the selected layout, which the user may then modify (e.g., via corners 412) if desired. Although not shown, third customize layout screen 400C may include additional saved layouts, and the user may scroll through the additional saved layouts (e.g., via a scroll bar).

Third customize layout screen 400C may also include input selection icon 414, for example, positioned on a portion (e.g., a middle portion) of each of first, second, and third image or video display tiles 424, 426, 428. As discussed above, each input selection icon 414 may include indication 416 of the currently selected input (e.g., input 1 for first image or video display tile 424, input 2 for second image or video display tile 426, and input 3 for third image or video display tile 428, as shown in FIG. 4C). As mentioned above, computing system 104 may only provide an input as an optional selection if a medical device is properly connected to the respective input of computing system 104. Additionally, input selection icons 414 may also include selection change icon 418, as discussed above. Third customize layout screen 400C may also include one or more of cancel icon 420 and/or save icon 422, as discussed above. Saving a customized layout may cause computing system 104 to display the saved layout as a saved layout at a later time (e.g., instead of or in addition to one of saved layouts 440A, 440B, 440C).

Furthermore, although not shown, user interface 130 may include additional customize layout screens for a varying number of inputs. For example, user interface 130 may include a customize layout screen for first, second, third, fourth, or more image or video display tiles (e.g., for four or more inputs). Alternatively or additionally, as mentioned above, user interface 130 may also provide for one or more of the image or video display tiles to display one or more previously saved images and/or videos, for example, along with the images and/or videos received from one or more input devices.

FIG. 5 illustrates an end or summary screen 500 of user interface 130 that may be displayed on touch screen 200. For example, as discussed above, selecting end examination icon 322 (FIG. 3) may cause computing system 104 to transition user interface 130 to end or summary screen 500. End or summary screen 500 may include information bar 202, as discussed above. Additionally, end or summary screen 500 may include a procedure summary section 502, which may include a patient name 504, a patient identification number 506, a patient age 508. Additionally, procedure summary section 502 may include an edit icon 510, for example, to allow a user to correct, update, or otherwise change information in procedure summary section 502. Furthermore, procedure summary section 502 may include a date section 512 indicating the date and/or day of the week for the procedure. Procedure summary section 502 may also include a procedure duration section 514, for example, indicating the procedure start time and/or the procedure end time. Procedure summary section 502 may also include a devices connected summary portion 516, for example, indicating the devices that have been used (e.g., were connected to computing system 104) during the procedure (e.g., Device 1 and Device 2, as shown in FIG. 5).

Moreover, end or summary screen 500 may include a video summary section 520 and/or an image summary section 522, for example, listing icons or tiles for each of the videos recorded and/or images captured during the procedure. The user may select the video and/or image icons or tiles to view a larger version of the video and/or image, for example, on touch screen 200 and/or on one or more display devices 106 (FIG. 1). End or summary screen 500 may also include a see all video icon 520A and/or a see all image icon 522A, for example, respectively selectable to view or display all of the videos recorded and/or images captured during the procedure. Furthermore, each video icon or tile may include a duration 524, for example, indicating the time or length of the video (e.g., as controlled via record icon 386). Each image icon or tile may include a time stamp 526, for example, indicating the time of day at which the image was captured (e.g., via image capture icon 382).

In some aspects, end or summary screen 500 may include an export icon 530. For example, the user selecting export icon 530 may cause computing system 104 to export, send, or transmit the video(s), image(s), and/or other information for the procedure to one or more memory devices or data storage systems (e.g., wired or wirelessly), via email, via Bluetooth, etc.

In some aspects, end or summary screen 500 may include a delete icon 540. For example, the user selecting delete icon 540 may cause computing system 104 to delete or remove the video(s), image(s), and/or other information for the procedure. Alternatively, the user may select one or more videos and/or images, and selecting delete icon 540 may delete those videos and/or images (e.g., a subset of the videos and/or images from the procedure). In some aspects, selecting delete icon 540 may cause touch screen 200 to display, via user interface 130, a confirmatory question for the user (e.g., “Are you sure you want to delete this procedure?”), which may help to ensure that the user does not inadvertently delete vides(s), image(s), and/or other information for the procedure. Alternatively or additionally, in some aspects, the user may have to select (e.g., touch) delete icon 540 for a period of time before user interface 130 deletes the video(s), image(s), and/or other information.

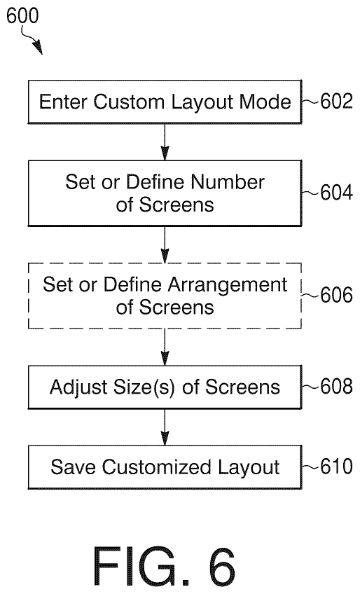

FIG. 6 depicts an example process 600 for establishing or otherwise setting a customized layout or display of one or more image(s), video(s), and/or data, for example, for medical device 102 of medical system 100 and/or other information. In some examples, one or more steps or decisions of process 600 may be performed by processor(s) 142 of computing system 104 of medical system 100.

Although not shown in FIG. 6, process 600 may include one or more optional preliminary steps that include detecting a connection of medical device 102 (and/or one or more other medical devices or inputs) to computing system 104. Upon connection, computing system 104 may perform a series of initialization steps to help establish data communication between computing system 104 and medical device 102, perform various checks, and/or set up operation parameters for various components of medical device 102, among other examples.

At a step 602, process 600 includes entering a custom layout mode. For example, step 602 may include computing system 104 receiving a selection of custom layout icon 338, for example, on active procedure screen 300 (FIG. 3). Upon receiving the selection, computing system 104 may transition or otherwise cause display of one or more of customize layout screens 400A, 400B, 400C (FIGS. 4A-4C) of user interface 130 on touch screen 200 to facilitate generation of a custom layout.

Next, at a step 604, process 600 includes setting or defining a number of screens. For example, step 604 may include computing system 104 receiving a selection of a number of screens based on input received via number of screen(s) control portion 402 on one or more of customize layout screens 400A, 400B, 400C. The number of screens selected may be the number of screens set or defined by computing system 104 for the custom layout. As mentioned, number of screen(s) control portion 402 may include display icon 404, add screen icon 406, and reduce screen icon 408. Manipulation of add screen icon 406 and reduce screen icon 408 (e.g., by the user) may cause computing system 104 to adjust the number displayed in display icon 404, while also transitioning between one or more of customize layout screens 400A, 400B, 400C.

At an optional step 606, process 600 may include an optional step that includes setting or defining an arrangement of the tiles screen(s). For example, as discussed above, the user may drag and drop each of the tiles or screen(s) to adjust positions and/or layout of the screen(s). In some aspects, the user may select an input for each of the one or more screens, for example, such that input 1 corresponds to first image or video display tile 424 and input 2 corresponds to second image or video display tile 426. In some aspects, the user may switch or otherwise adjust which input corresponds to first image or video display tile 424 or second image or video display tile 426. Moreover, in some aspects, customize layout screens 400A, 400B, 400C may include one or more previously saved or pre-programmed arrangements (e.g., 430A-430C and 440A-440C), and the user may select one or more of these arrangements. Additionally, the user may modify one or more of the previously saved or pre-programmed arrangements, for example, by dragging and dropping the respective tiles or icons. In these aspects, computing system 104 may receive the one or more inputs indicative of these selections and/or adjustments to optionally set or define the arrangement of the one or more screens.

Additionally, at a step 608, process 600 includes adjusting a size of the one or more screens. For example, as discussed above, each of customize layout screens 400A, 400B, 400C and their respective image or video tiles each include corners 412, which may allow the user to adjust the size and/or shape of the respective number of screens. As mentioned, in some aspects, customize layout screens 400A, 400B, 400C may include one or more previously saved or pre-programmed arrangements (e.g., 430A-430C and 440A-440C), and the user may select one or more of these arrangements. Additionally, the user may modify one or more of the previously saved or pre-programmed arrangements, for example, via one or more of corners 412. In these aspects, computing system 104 may receive one or more inputs indicative of these selections and/or adjustments to adjust the size of the one or more screens.

Moreover, at a step 610, process 600 includes saving a customized layout of the one or more screens. For example, as discussed above, each of customize layout screens 400A, 400B, 400C include save icon 422. Upon receiving a selection of save icon 422, computing system 104 may save the selected number, size, shape, and/or arrangement of the respective number of screens as a custom layout. Additionally, in some aspects, computing system 104 may cause active procedure screen 300 to display the saved custom layout as a pre-defined layout, for example, in layout selection bar 330. Furthermore, the selection and/or saving of the custom layout may include a designation of one or more medical devices (e.g., where the respective image, video, and/or other data will be displayed), a designation of the type of procedure, the user (e.g., the name of the doctor or other medical professional), and/or one or more other designations.

Furthermore, the custom layout may be used during a medical procedure. For example, image and/or video tiles of the user-specified shape, size, and/or arrangement within the custom layout are populated with image and/or video data from respective inputs to computing system 104 for display, for example, via display device(s) 106. The respective inputs correspond to or are associated with the image and/or video tiles.

It is noted that one or more steps of process 600 may be repeated, for example, before and/or during a medical procedure. For example, the user may adjust a previously saved custom layout during a procedure, and may then save the adjusted layout as a new custom layout or replace the previously saved custom layout. Additionally, this selection, manipulation, and saving of the customized layout may be performed before a procedure or during a procedure, and the saved customized layout may be used for a future procedure as well.

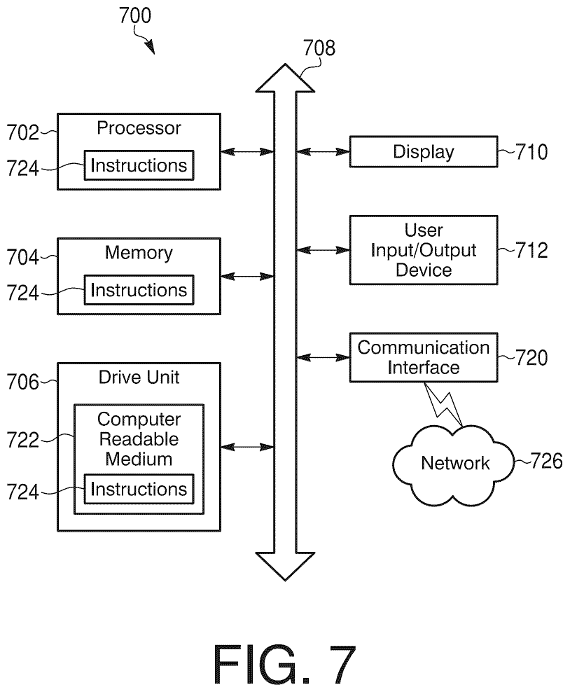

FIG. 7 depicts an example of a computer 700. FIG. 7 is a simplified functional block diagram of computer 700 that may be configured as a device for executing processes, steps, or operations depicted in, or described with respect to, FIGS. 2A-6 and, according to exemplary embodiments of this disclosure. For example, computer 700 may be configured as one or more of medical device 102, computing system 104, display device(s) 106, optional server side system(s) 108, and/or another device or component according to exemplary embodiments of this disclosure. In various embodiments, any of the systems herein may be or include computer 700 including, e.g., a data communication interface 720 for packet data communication. Computer 700 may communicate with one or more other computers, for example, using an electronic network 726 (e.g., via data communication interface 720). Electronic network 726 may include a wired or wireless network, for example, similar to optional network 110 depicted in FIG. 1.

Computer 700 also may include a central processing unit (“CPU”), in the form of one or more processors 702, for executing program instructions 724. In some examples, processors 702 may be or include one or more field-programmable gate arrays (FPGAs). Program instructions 724 may include at least instructions for performing usage monitoring (e.g., if computer 700 is or is included in computing system 104).

Computer 700 may include an internal communication bus 708. Computer 700 may also include a drive unit 706 (such as read-only memory (ROM), hard disk drive (HDD), solid-state disk drive (SDD), etc.) that may store data on a computer readable medium 722 (e.g., a non-transitory computer readable medium), although computer 700 may receive programming and data via network communications. Computer 700 may also have a memory 704 (such as random-access memory (RAM)) storing instructions 724 for executing techniques presented herein. It is noted, however, that in some aspects, instructions 724 may be stored temporarily or permanently within other modules of computer 700 (e.g., processor 702 and/or computer readable medium 722). Computer 700 also may include user input and output devices 712 and/or a display 710 to connect with input and/or output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may, at times, be communicated through the Internet or various other telecommunication networks. Such communications, e.g., may enable loading of the software from one computer or processor into another. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A computing system configured to be removably coupled to one or more medical devices, the computing system comprising:
at least one memory configured to store instructions; and
at least one processor configured to execute the instructions to:
detect the one or more medical devices that are removably coupled to the computing system, each of the one or more medical devices configured to provide image or video data to the computing system;
cause display of a user interface associated with generation of a custom layout for presenting the image or video data provided by the one or more medical devices, the user interface including one or more image or video tiles configurable for inclusion in the custom layout;
receive one or more inputs via the user interface that define the custom layout, wherein the one or more inputs include one or more of: a number of the one or more image or video tiles, a size of the one or more image or video tiles, or an association of the one or more medical devices with the one or more image or video tiles; and
store the custom layout such that, when the image or video data is received from the one or more medical devices, the image or video data is provided for display in accordance with the custom layout to one or more display devices associated with the computing system.

2. The computing system of claim 1, wherein the computing system includes a touch screen configured to display the user interface and receive the one or more inputs.

3. The computing system of claim 2, wherein each of the one or more image or video tiles include a plurality of frame edges, and wherein each of the plurality of frame edges are movable to adjust a size and/or shape of the image or video tile.

4. The computing system of claim 3, wherein the user interface includes a number of screens control portion that is manipulatable to adjust a number of the one or more image or video tiles included in the custom layout.

5. The computing system of claim 4, wherein the number of screens control portion includes an add screen icon and a reduce screen icon that are each selectable to adjust the number of the one or more image or video tiles included in the custom layout.

6. The computing system of claim 3, wherein each of the one or more image or video tiles includes an input selection icon that includes an indication of the association of a medical device, from the one or more medical devices removably coupled to the computing system, with the respective image or video tile, and wherein the association causes image or video data received from the medical device to be displayed via the respective image or video tile in the custom layout.

7. The computing system of claim 6, wherein each input selection icon further includes a selection change icon that is selectable to adjust which medical device, from the one or more medical devices removably coupled to the computing system, is associated with the respective image or video tile.

8. The computing system of claim 3, wherein each of the one or more image or video tiles is movable, via the user interface, to adjust a position of the respective image or video tile included in the custom layout.

9. The computing system of claim 3, wherein the user interface is further configured to display a plurality of saved layouts of the one or more image or video tiles.

10. The computing system of claim 3, wherein the user interface further includes patient and/or user information.

11. The computing system of claim 10, wherein the user interface includes an active procedure screen that includes the custom layout selected for a procedure to be performed using the one or more medical devices, and one or more of: an image capture icon that is selectable to capture one or more still images received from the one or more medical devices, a record icon that is selectable to record one or more videos received from the one or more medical devices, and a stream icon that is selectable to stream one or more aspects of the procedure.

12. The computing system of claim 2, wherein the one or more medical devices include one or more imaging or video devices, and one or more illumination devices controllable by the computing system, and wherein the user interface is configured to display an illumination control section that receives user inputs to adjust a brightness of the one or more illumination devices.

13. The computing system of claim 12, wherein the illumination control section includes a slider that is movable within a track to adjust the brightness of the one or more illumination devices.

14. The computing system of claim 12, wherein the computing system is coupled to each of the one or more medical devices via a wired connection through an umbilicus.

15. The computing system of claim 1, wherein the computing system is coupled to the one or more display devices via a wired connection or via a wireless connection.

16. A medical computing system, comprising:
a display for displaying a plurality of user interfaces, and configured to receive one or more inputs via the plurality of user interfaces;
at least one memory configured to store instructions; and
at least one processor configured to execute the instructions to:
detect one or more input sources that are coupled to the medical computing system, each of the one or more input sources configured to provide image or video data to the medical computing system;
cause display of a user interface, from the plurality of user interfaces, associated with generation of a custom layout for presenting image or video data provided by the one or more input sources, the user interface including one or more image or video tiles configurable for inclusion in the custom layout;
receive one or more inputs via the user interface that define the custom layout, wherein the one or more inputs includes one or more of: a number of one or more image or video tiles, a size of the one or more image or video tiles, or an association of the one or more input sources with the one or more image or video tiles; and
store the custom layout such that, when the image or video data is received from the one or more input sources, the image or video data is provided for display in accordance with the custom layout to one or more display devices associated with the computing system.

17. The medical computing system of claim 16, wherein each of the one or more image or video tiles include a plurality of L-shaped corners, and wherein each of the plurality of L-shaped corners are movable to adjust a size and/or shape of the corresponding image or video tile.

18. The medical computing system of claim 17, wherein each of the one or more custom layouts includes a number of screens control portion that is adjustable to adjust a number of image or video tiles on the custom layout, and wherein the number of screens control portion includes an add screen icon and a reduce screen icon that are each selectable to adjust the number of image or video tiles on the custom layout.

19. A method performed by a computing system that is removably connectable to a medical device for use during a medical procedure, the method comprising:
receiving an input, via a user interface displayed on a display of the computing system, to enter a custom layout mode, wherein, in the custom layout mode, the user interface displays one or more image or video tiles that each include a plurality of frame edges that are manipulatable to adjust a size of each of the image or video tiles;
receiving, via the user interface, one or more inputs of a number of screens corresponding to the number of image or video tiles;
receiving, via the user interface, one or more inputs to adjust the size of one or more of the image or video tiles; and
receiving, via the user interface one or more inputs to save the size and/or shape of the one or more image or video tiles as a customized layout.

20. The method of claim 19, further comprising, receiving one or more inputs to adjust an arrangement of the one or more image or video tiles on the user interface.
